(19) United States
(12) Patent Application Publication
(10) Pub. No.: US 2010/0119592 A1
(43) Pub. Date: May 13, 2010

(54) CYTARABINE FOR TREATMENT OF GLIOMA

(76) Inventor: Bruce Michael Frankel, Mount Pleasant, SC (US)

Correspondence Address: PATENT CORRESPONDENCE;ARNALL GOLDEN GREGORY LLP, 171 17TH STREET NW, SUITE 2100, ATLANTA, GA, 30363, US

(21) Appl. No.: 12/518,235

(22) PCT Filed: Dec. 7, 2007
(86) PCT No.: PCT/US07/25115

§ 371 Date: Jan. 13, 2010

Related U.S. Application Data
(60) Provisional application No. 60/873,609, filed on Dec. 8, 2006.

(51) Int. Cl. A61K 9/127; A61K 31/7008; A61P 35/00

(52) U.S. Cl. 424/450; 514/49

(57) ABSTRACT

Provided are methods and compositions for the treatment of a subject with glioma. An exemplary method can comprise administering cytarabine to the subject. For example, DepoCyt® can be administered to the subject. An exemplary composition can comprise cytarabine. For example the composition can comprise DepoCyt®.

### CROSS-REFERENCE TO RELATED APPLICATIONS

This application claims the benefit of U.S. provisional application No. 60/873,609 filed on Dec. 8, 2006. The aforementioned application is herein incorporated by this reference in its entirety.

### BACKGROUND

Despite significant improvements in diagnostic imaging and neurosurgical techniques, the current treatment modalities for high-grade gliomas are inadequate. As such, the median survival for patients with GBM is 12-18 months, even after cytoreductive surgery, radiation and chemotherapy (1). Furthermore, the outlook for patients with these tumors has not changed significantly in the twentieth century with a 5-year survival rate of 10%, and a mortality rate of nearly 100% (2, 3). Recurrence is nearly inevitable and usually occurs in the form of local contiguous growth within a 2 cm margin of resection (4).

The median interval from initial diagnosis to tumor recurrence, either clinical or radiographic, is 4.9 months (5). Median survival rates following GBM recurrence are variable. Either re-resection or systemic chemotherapy alone yields a median survival of 14 (6), and 24 weeks (7), respectively. Re-resection followed by systemic chemotherapy increases median survival to approximately 36 weeks (5). Other adjuvant modalities including use of local chemotherapy (BCNU wafers), or interstitial brachytherapy (GliaSite) has demonstrated a median survival of 31 weeks (8, 9), and 36 weeks (10), respectively.

Needed in the art are safe and efficacious glioma anti-cancer therapies.

### SUMMARY

In accordance with the purposes of this invention, as embodied and broadly described herein, this invention, in one aspect, relates to methods for the treatment of a subject with glioma. An exemplary method can comprise administering cytarabine to the subject. For example, DepoCyt® can be administered to the subject. Also provided are compositions for the treatment of a subject with glioma. An exemplary composition can comprise cytarabine. For example, the composition can comprise DepoCyt®.

Additional advantages will be set forth in part in the description which follows, and in part will be obvious for the description, or may be learned by practice of the aspects described below. The advantages described below will be realized and attained by means of the elements and combinations particularly pointed out in the appended claims. It is to be understood that both the forgoing general description and the following detailed description are exemplary only and are not restrictive.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows gadolinium enhanced MR image (Case 1) demonstrating recurrent left frontal GBM and right periventricular enhancement. H&E stains are shown from biopsies of both regions with corresponding Ki-67 immunohistochemistry (upper insets) (11), and nestin immunocytochemistry staining (lower insets) (12).

FIG. 2 shows gadolinium enhanced MR images (Case 1) comparing pre- (A) and post-DepoCyt® treatment (B) after completion of the consolidation phase (cycle 6). After four months of treatment, the original left frontal lesion, and periventricular enhancement has disappeared.

FIG. 3 shows gadolinium enhanced MR image (Case 2) demonstrating recurrent GBM and bilateral periventricular enhancement. Biopsies of both regions with corresponding Ki-67 immunohistochemistry (upper images) (11), and nestin immunocytochemistry staining (lower images) (12) are shown.

FIG. 4 shows gadolinium enhanced MR images (Case 2) comparing pre- (A) and post-DepoCyt® treatment (B) after completion of the induction phase (cycle 2). After one month of treatment, periventricular enhancement has significantly diminished.

### DESCRIPTION OF THE PREFERRED EMBODIMENTS

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a composition” or “a catheter” includes two or more such compositions or catheters.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings: “Optional” or “optionally” means that the subsequently described event or circumstance may or may not occur, and that the description includes instances where said event or circumstance occurs and instances where it does not. For example, the phrase “optionally a steroid can be used” means that a described method may or may not utilize a steroid and that the description includes both methods utilizing a steroid and methods not utilizing a steroid.

As used throughout, by a “subject” is meant an individual. The subject may be a vertebrate, more specifically a mammal (e.g., a human, horse, pig, rabbit, dog, sheep, goat, non-human primate, cow, cat, guinea pig or rodent), a fish, a bird, a reptile or an amphibian. The term does not denote a particular age or sex. Thus, adult and newborn subjects, as well as fetuses, whether male or female, are intended to be covered. A patient refers to a subject afflicted with a disease or disorder, such as a glioma, for example, a glioblastoma. The term “patient” includes human and veterinary subjects.

Provided herein are methods for the treatment of a subject with glioma. “Treatment”, “treating”, or “therapy” means to administer a composition or combination of compositions to a subject with a glioma. The effect of the administration to the subject can have the effect of, but is not limited to, reducing the symptoms of the glioma, a reduction in the severity of the glioma, or the complete ablation of the glioma.

The glioma to be treated can be Glioblastoma Multiforme or recurrent Glioblastoma Multiforme. The glioma to be treated can also be selected from the group consisting of astrocytoma grade I, astrocytoma grade II, astrocytoma grade III, astrocytoma grade IV, ependymoma, oligodendroglioma, brainstem glioma, and mixed glioma (for example, oligoastrocytoma and ganglioglioma). However, any glioma can be treated using the disclosed methods and compositions. Thus, the effect of the administration to the subject can reduce the symptoms of the glioma, lead to a reduction in the severity of the glioma, or lead to the complete ablation of the glioma in the subject. The cytarabine, including DepoCyt® can also be used as a radiosensitizer. As used herein, “radiosensitizer” means any treatment that when added to radiation therapy results in a combination therapy that has a greater therapeutic effect than the radiation therapy alone.

When used in described methods or other treatments, or in the pharmaceutical formulations, or compositions disclosed herein, an “effective amount” of one of the disclosed compounds can be employed in pure form or, where such forms exist, in pharmaceutically acceptable salt form, and with or without a pharmaceutically acceptable excipient, carrier, or other additive.

The specific effective amount for any particular subject will depend upon a variety of factors including the disorder being treated and the severity of the disorder; the identity and activity of the specific composition employed; the age, body weight, general health, sex and diet of the patient; the time of administration; the route of administration; the rate of excretion of the specific composition employed; the duration of the treatment; drugs used in combination or coincidental with the specific composition employed and like factors well known in the medical arts.

For example, it is well within the skill of the art to start doses of a composition at levels lower than those required to achieve the desired therapeutic effect and to gradually increase the dosage until the desired effect is achieved. One can also evaluate the particular aspects of the medical history, signs, symptoms, and objective laboratory tests that are known to be useful in evaluating the status of a subject in need of attention for a glioma. These signs, symptoms, and objective laboratory tests will vary, depending upon the particular glioma or condition being treated or prevented, as will be known to any clinician who treats such patients or a researcher conducting experimentation in this field.

For example, if, based on a comparison with an appropriate control group and/or knowledge of the normal progression of the disease in the general population or the particular individual: 1) a subject's physical condition is shown to be improved (e.g., a tumor has partially or fully regressed), 2) the progression of the disease or condition is shown to be stabilized, or slowed, or reversed, or 3) the need for other medications for treating the disease or condition is lessened or obviated, then a particular treatment regimen will be considered efficacious. If desired, the effective daily dose can be divided into multiple doses for purposes of administration. Consequently, single dose compositions can contain such amounts or submultiples thereof to make up the daily dose. Thus, a treatment session can comprise multiple dosing administrations to reach the administered effective amount for that treatment session.

The dosage can be adjusted by the individual physician or the subject in the event of any counterindications. Dosage can vary, and can be administered in one or more dose administrations daily, for one or several days. Guidance can be found in the literature for appropriate dosages for given classes of pharmaceutical products.

A disclosed method can comprise administering to the subject an effective amount of cytarabine and a diuretic. A “diuretic” means any drug that elevates the rate of bodily urine excretion (diuresis), and thereby increases the excretion of water from the body. In one aspect, the diuretic is acetazolamide (Diamox®). In another aspect, the diuretic is furosemide. For example, Diamox® can be administered in a dose range from about 250 mg to about 2.5 grams/day divided into b.i.d to q.i.d. dosing. Such administration can be varied based on the administration considerations described herein. In one aspect, daily dosed diuretic can be determined based on intracranial pressure (ICP) measurements. In a further aspect, at least about 250 mg of Diamox can be administered. For example, about 250, 500, 1000, 1250, 1500, 1750, 2000, 2250, or 2500 mg of Diamox can be administered with the Depocyt®.

Other non-limiting examples of diuretics include, organomercurials, such as Chlormerodrin, Meralluride, Mercamphamide, Mercaptomerin Sodium, Mercumallylic Acid, Mercumatilin Sodium, Mercurous Chloride and Mersalyl; Pteridines such as Furterene and Triamterene; Purines such as Acefylline, 7-Morpholinomethyltheophylline, Pamabrom, Protheobromine and Theobromine; Steroids such as Canrenone, Oleandrin and Spironolactone; Sulfonamide derivatives such as Acetazolmide, Ambuside, Azosemide, Bumetanide, Butazolamide, Chloraminophenamide, Clofenamide, Clopamide, Clorexolene, Diphenylmethane-4.4′-disulfonamide, Disulfamide, Ethoxzolamide, Furosemide, Indapamide, Mefruside, Methazolamide, Piretanide, Quinethazone, Torsemide, Tripamide and Xipamide; Uracils such as Aminometradine and Amisometradine; and others such as Amanozine, Amiloride, Arbutin, Chlorazanil, Ethacrynic Acid, Etozolin, Hydracarbazine, Isosorbide, Mannitol, Metochalcone, Muzolimine, Perhexiline, Ticrynafen and Urea.

If used, a diuretic, or combinations thereof, can be administered in a variety of ways. For example, the diuretic can be administered orally. The diuretic can also be administered intravenously, intrathecally, or intraventricularly. The diuretic or combination thereof can be administered before, concurrently or following administration of cytarabine. Similarly, other compositions described herein, including steroids, cytarabine, DepoCyt®, and diuretics can be administered in a number of ways. Administration may be topically (including ophthalmically, vaginally, rectally, intranasally), intrathecially, intraventricularly, orally, by inhalation, or parenterally, for example by intravenous drip, intravenous injection, subcutaneous, intraperitoneal or intramuscular injection.

Further provided are methods for the treatment of a subject with glioma comprising administering an effective amount of cytarabine and one or more other anti-cancer drugs or therapies, or combinations thereof. As used herein, “anti-cancer drug” means any drug useful for the purpose of treating cancer or cancer metastasis, for example by reducing the symptoms of the cancer, a reduction in the severity of the cancer, or the complete ablation of the cancer.

Exemplary anti-cancer drugs that can be used in the disclosed methods can be selected from the group consisting of temozolamide, Gliadel wafer, an anti-VEGF antibody or antibody fragment, a small molecule VEGF inhibitor, irinotecan, BCNU, CCNU, PCV therapy, retinoic acid, thalidomide, Tamoxifen, targeted immunotoxin, immunomodulator, and an inhibitor to the FGF, EGF, PDGF, mTor, AKT or Ras biochemical pathway.

Other non limiting examples of anti-cancer drugs include, Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131;Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

In another aspect, the anti-cancer therapy comprises radiotherapy or interstitial brachytherapy.

Also disclosed are methods comprising administering an effective amount of cytarabine in a predetermined fluid volume into one or more ventricles of the subject's brain. The effective amount of cytarabine can be allowed to remain within the subject to effect treatment of the glioma. In yet another exemplary aspect, disclosed methods can comprise intrathecally administering an effective amount of cytarabine and allowing the effective amount of cytarabine to remain within the subject to treat the glioma. “Remain in the subject” is not intended to imply that the administered compositions necessarily forever remain in the subject, as such compositions may eventually be removed, for example by normal metabolic processes within the body. Thus, “remain” is intended to describe methods wherein the administered compound or compounds are not intentionally removed from the body by one administering the therapeutic protocol to the subject.

The administered effective amount of cytarabine in a predetermined fluid volume can increase intracranial pressure. For example, in one aspect, the cytarabine and predetermined fluid volume is not purposefully withdrawn from the subject and therefore the administration can increase the intracranial pressure of the subject. In one aspect, intracranial pressure can be increased to at least about 5 cm H₂O. In other aspects, intracranial pressure can be increased to about 10, 15, 20, 25, 30, 35, 40, 45, 50, or 55 cm H₂O. Measurements of intracranial pressure are routinely made by those of skill in the art.

In the disclosed methods, the cytarabine can be DepoCyt® (Pacira Pharmaceuticals, San Diego, Calif.). DepoCyt® is a sterile, injectable suspension of the antimetabolite cytarabine, encapsulated into multivesicular lipid-based particles. Cytarabine is also known as 4-amino-1-beta-D-arabinofuranosyl-2(1H)-pyrimidinone or cytosine arabinoside (C₉H₁₃N₃0₅, molecular weight 243.22). DepoCyt® is available in 5 mL, ready-to-use, single-use vials containing 50 mg of cytarabine. DepoCyt® is formulated as a sterile, non-pyrogenic, white to off-white suspension of cytarabine in Sodium Chloride 0.9% w/v in Water for Injection. DepoCyt® is preservative-free. Cytarabine, the active ingredient, is present at a concentration of 10 mg/mL, and is encapsulated in the particles. Inactive ingredients at their respective approximate concentrations are cholesterol, 4.1 mg/mL; triolein, 1.2 mg/mL; dioleoylphosphatidylcholine (DOPC), 5.7 mg/mL; and dipalmitoylphosphatidylglycerol (DPPG), 1.0 mg/mL. The pH of the product falls within the range from 5.5 to 8.5.

DepoCyt®'s mechanism of action is a sustained-release formulation of the active ingredient cytarabine designed for direct administration into the cerebrospinal fluid (CSF). Cytarabine is a cell cycle phase-specific antineoplastic agent, affecting cells only during the S-phase of cell division. Intracellularly, cytarabine is converted into cytarabine-5′-triphosphate (ara-CTP), which is the active metabolite. The mechanism of action is not completely understood, but it appears that ara-CTP acts primarily through inhibition of DNA polymerase. Incorporation into DNA and RNA may also contribute to cytarabine cytotoxicity. Cytarabine is cytotoxic to a wide variety of proliferating mammalian cells in culture.

The effective amount of DepoCyt® or cytarabine can be administered during one or more treatment sessions. The sum dosage of DepoCyt® or cytarabine administered over all treatment sessions can be at least about 20 milligrams. For example, the sum dosage administered over all treatment sessions can be at least about 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, or 750 milligrams or greater. In one aspect, the sum dosage of DepoCyt® or cytarabine administered over all treatment sessions is greater than about 450 milligrams.

The effective amount of DepoCyt® or cytarabine administered to the subject at each treatment session can be at least about 20, 30, 40, or 50 milligrams or greater. The effective amount of DepoCyt® can be administered in solution. For example, the effective amount of DepoCyt® can be administered in a solution having a concentration of about 20 milligrams DepoCyt® or less in each milliliter of solution. In another aspect, the effective amount of DepoCyt® can be administered in a solution having a concentration of about 10 milligrams of DepoCyt® or less in each milliliter of solution. Provided herein is a container comprising 5, 10, 20, 30, or 40 milligrams of Depocyt®.

One or more treatment sessions can be completed in at least about 1, 2, 3, 4, 5, 6, 7, 8, 9, 10, 11, 12, 13, 14, or 15 months or longer. In one aspect, the treatment sessions can comprise administering at least about 20 milligrams of DepoCyt® to the subject at each of a first and second treatment sessions. The first and second treatment sessions can be temporally separated by about two weeks. The treatment sessions can also comprise administering at least about 20 milligrams of DepoCyt® to the subject at each of a third, fourth, fifth and sixth treatment session. Each of the third, fourth, fifth and sixth treatment sessions can be temporally separated by at least about two weeks. In one aspect, the fifth and sixth treatment sessions are temporally separated by about four weeks. The fifth and sixth can be followed by treatment sessions comprises administering at least about 20 milligrams of DepoCyt® to the subject at a seventh, eighth, ninth, tenth, eleventh, twelfth, thirteenth, and fourteenth or more treatment session. Each of the seventh, eighth, ninth, tenth, eleventh, twelfth, thirteenth, and fourteenth or more treatment sessions can be temporally separated by about four weeks.

In one aspect, greater than 9 sessions of about 50 milligrams of DepoCyt® are administered to the subject. The administration of each session can be separated by about two to four weeks.

Cytarabine, including DepoCyt® can be administered intrathecally or intraventricularly as described above. For example, the DepoCyt® can be administered directly by an intrathecal catheter attached to a subcutaneous port, by spinal puncture needle, or by temporary catheter placement; or by an Ommaya reservoir, a ventricular shunt, or ventricular shunt reservoir. The DepoCyt® or cytarabine can also be injected directly into the tumor or surrounding parenchyma using an implanted reservoir catheter system placed into the tumor or resected tumor bed. The Implanted reservoir catheter systems useful for such administration are known to those skilled in the art. In yet another aspect, the DepoCyt® or cytarabine can be directly infused into the tumor, resected tumor bed, or surrounding brain parenchyma using a catheter attached to an implanted or external pump or the DepoCyt® or cytarabine can be infused by convection enhanced delivery. An amount of spinal fluid can be removed prior to administration of DepoCyt® or cytarabine by any of the disclosed methods. An intrathecal catheter attached to a subcutaneous port as one device can be used for intrathecal administration of the described compounds and in the disclosed methods.

Also provided are compositions comprising a mixture of cytarabine and a diuretic. In one aspect, an exemplary composition comprises a mixture of DepoCyt® and a diuretic. The mixture of DepoCyt® and a diuretic can comprise at least 5 mg of DepoCyt®. For example, the composition can comprise a mixture of about 10, 15, 20, 25, 30, 35, 40, 45, 50, 55, 60, 65, 70, 75, 80, 85, 90, 95, or 100 mg of DepoCyt® and a diuretic. In a further aspect, a composition comprising a mixture of DepoCyt® and a diuretic, wherein the diuretic is acetazolamide (Diamox), wherein the mixture comprises at least about 250 mg of Diamox. For example, the composition can comprise a mixture of about 250, 500, 1000, 1250, 1500, 1750, 2000, 2250, or 2500 mg of Diamox.

The above methods can optionally comprise administration of an effective amount of a steroid before, during, or after administration of a cytarabine formulation.

The compounds and methods can include a combination with one or more second active agents. Second active agents can be large molecules (e.g., proteins) or small molecules (e.g., synthetic inorganic, organometallic, or organic molecules). Examples of large molecule active agents include, but are not limited to, hematopoietic growth factors, cytokines, and monoclonal and polyclonal antibodies. Specific examples of the active agents are anti-CD40 monoclonal antibodies (such as, for example, SGN-40); histone deacetlyase inhibitors (such as, for example, SAHA and LAQ 824); heat-shock protein-90 inhibitors (such as, for example, 17-AAG); insulin-like growth factor-1 receptor kinase inhibitors; vascular endothelial growth factor receptor kinase inhibitors (such as, for example, PTK787); insulin growth factor receptor inhibitors; lysophosphatidic acid acyltransrerase inhibitors; IkB kinase inhibitors; p38MAPK inhibitors; EGFR inhibitors (such as, for example, gefitinib and erlotinib HCL); HER-2 antibodies (such as, for example, trastuzumab (Herceptin®) and pertuzumab (Omnitarg™)); VEGFR antibodies (such as, for example, bevacizumab (Avastin™)); VEGFR inhibitors (such as, for example, flk-1 specific kinase inhibitors, SU5416 and ptk787/zk222584); P13K inhibitors (such as, for example, wortmannin); C-Met inhibitors (such as, for example, PHA-665752); monoclonal antibodies (such as, for example, rituximab (Rituxan®), tositumomab (Bexxar®), edrecolomab (Panorex®) and G250); and anti-TNF-α antibodies. Specific second active compounds that can be combined with compounds of this invention vary depending on the specific indication to be treated, prevented or managed. The disclosed compounds, including antibodies, can be administered intravenously, intraperitoneally, intramuscularly, subcutaneously, intracavity, intraventricularly, intrathecially, or transdermally.

Also, pharmaceutical formulations comprising the disclosed compounds are disclosed herein. A suitable pharmaceutical formulation can comprise any of the disclosed compounds with a pharmaceutically acceptable carrier. The disclosed pharmaceutical formulations can be used therapeutically or prophylactically.

By “pharmaceutically acceptable” is meant a material that is not biologically or otherwise undesirable, i.e., the material may be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy, 21^{st} Ed., Lippincott Williams & Wilkins, Philadelphia, Pa., 2005, which is incorporated by reference herein for its teachings of carriers and pharmaceutical formulations. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8 (e.g., from about 7 to about 7.5). Further carriers include sustained release preparations such as semipermeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, marine oils, and injectable organic esters such as ethyl oleate. Aqueous carriers include water, alcoholic/aqueous solutions, and emulsions or suspensions, including saline and buffered media. Parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations for topical administration may include ointments, lotions, creams, gels, drops, suppositories, sprays, liquids and powders. Conventional pharmaceutical carriers, aqueous, powder or oily bases, thickeners and the like can be desirable.

Pharmaceutical formulations for oral administration include, but are not limited to, powders or granules, suspensions or solutions in water or non-aqueous media, capsules, gel-caps, sachets, or tablets. Thickeners, flavorings, diluents, emulsifiers, dispersing aids, or binders can be desirable.

Some of the formulations can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Also disclosed are kits or packages of pharmaceutical formulations designed for use in the regimens described herein. An example of a kit can include one or more of the following items: DepoCyt® variable dosages (for example comprising 20 mg or more Depocyt®), spinal needle, manometer, butterfly needles, syringes, sterile field drape, betadine, alcohol pads, Diamox, and Decadron (4 mg t.i.d. for 5 days duration), and a sterile flush.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Case Reports

### Case 1

### History and Initial Examination

A 30-year old female presented with headaches, diplopia and a Karnofsky Performance Scale (KPS) of 70. Magnetic resonance images demonstrated a 2.5 cm left frontal mass enhancing mass with extensive surrounding edema and 1 cm subfalcine herniation.

### Treatments and Pathological Analyses

The patient underwent a left frontal craniotomy and gross total resection of the lesion. Pathology revealed an anaplastic gemistocytic astrocytoma. Surgery was followed by adjuvant external-beam radiation therapy (59 Gy) and concurrent 75 mg/m² temozolamide. She subsequently underwent 12 cycles of temozolamide to 200 mg/m². KPS improved to 100.

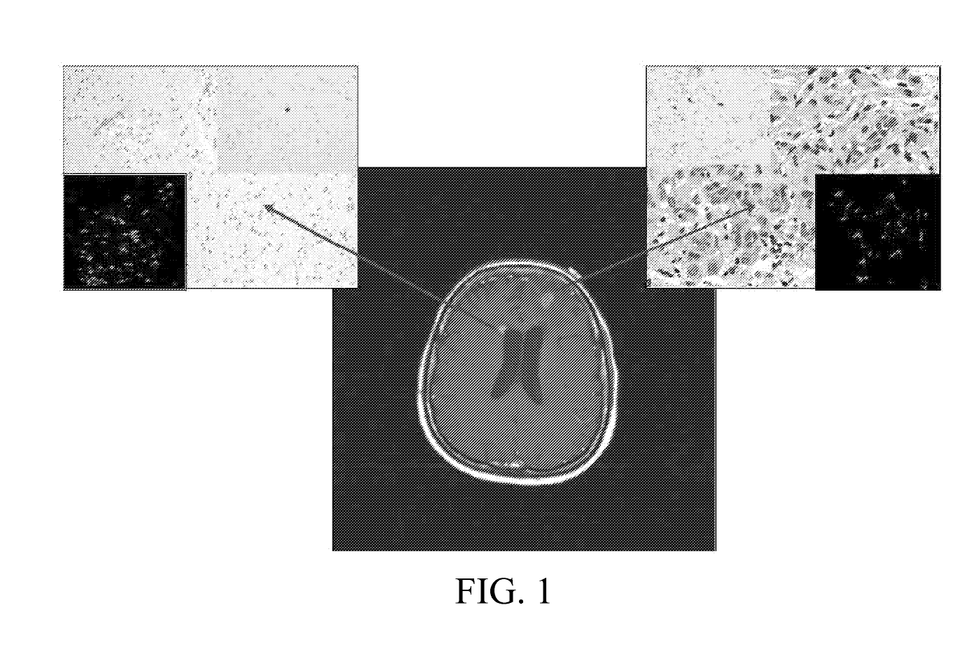

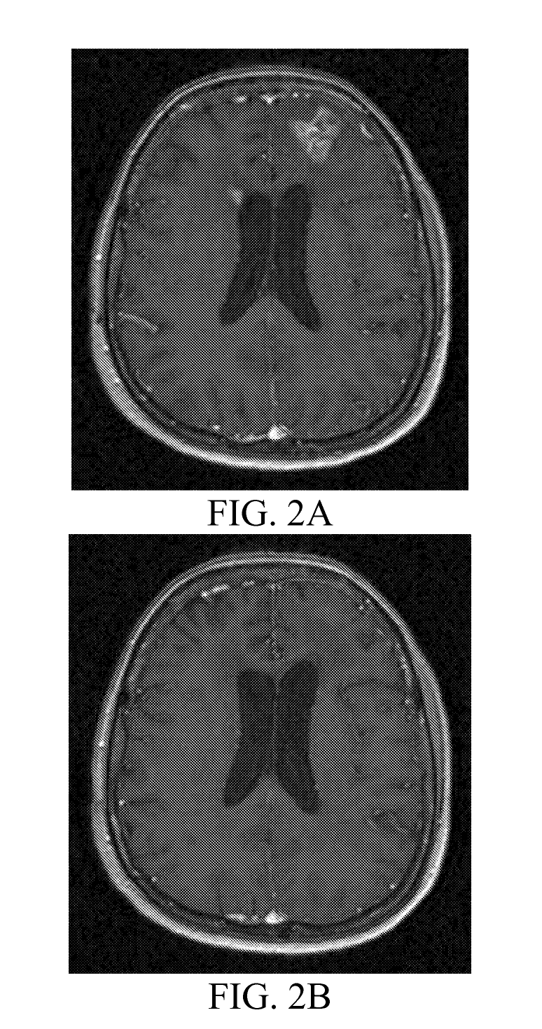

Fifty-four weeks later, MR imaging showed local tumor recurrence in the left frontal lobe and a new area of right-sided periventricular enhancement adjacent to the frontal horn of the lateral ventricle (FIGS. 1 and 2A). The patient's KPS was 90 and underwent biopsy of both the left-sided hemispheric lesion, right-sided periventricular lesion, and placement of Ommaya reservoir. Pathology results revealed left frontal GBM with a Ki-67 labeling index of 10%, and hypercellularity of the right frontal periventricular lesion with a Ki-67 labeling index of 1.5% (FIG. 1). Both biopsy specimens demonstrated intense nestin immunoreactivity (FIG. 1).

### Liposomal Encapsulated Ara-C (DepoCyt®) Treatment

After failing standard therapy with evidence of bi-hemispheric spread and progression to GBM, the off-label use of intrathecal liposomal encapsulated Ara-C (DepoCyt®) was discussed with the patient. DepoCyt®, a slow-release formulation of cytarabine (Ara-C), maintains cytotoxic concentrations of free Ara-C in the CSF for >14 days following a single injection (13).

A dosing regimen is known for treating neoplastic meningitis from solid tumors (11). In the exemplary DepoCyt® regimen for glioma patients undergo an induction phase of IT DepoCyt®, using 50 mg every two weeks for one month (Cycles 1, 2). MRI, clinical response, and systemic toxicity utilizing blood chemistry analysis are performed. Patients with stable disease, not exhibiting systemic toxicity, undergo a three month consolidation phase of IT DepoCyt®, using 50 mg every two weeks for one month, followed by 50 mg IT DepoCyt® every four weeks for two months (cycles 3-6). Evaluation of the therapeutic response is again assessed by MRI, clinical examination, and blood chemistry analysis. Those without progression or toxicity undergo further consolidation therapy using 50 mg IT DepoCyt® every four weeks for three months (cycles 7-9). Patients receive dexamethasone 4 mg TID on days 1-5 of each cycle.

The patient completed the induction (cycles 1, 2) and consolidation phases (cycles 3-6). She did not have radiographic or neurological progression and received an additional 5 cycles of consolidation therapy. The patient tolerated the treatment well, except for headache during the first three days of treatment. No other treatment-related complications occurred. She was disease free clinically and radiographically (including both right periventricular and left cortical areas), 36 weeks after beginning IT DepoCyt® for recurrent GBM (FIG. 2A, B).

### Case 2

### History and Initial Examination

A 47-year old male presented with headaches, and new onset of seizures with a Karnofsky Performance Scale (KPS) of 100. Magnetic resonance images demonstrated diffuse right frontal edema with a 1.8 cm area of enhancement on the right adjacent to the frontal horn of the lateral ventricle.

### Treatments and Pathological Analyses

The patient underwent a right frontal biopsy of the lesion. Pathology revealed anaplastic astrocytoma. Surgery was followed by adjuvant external-beam radiation therapy and concurrent 75 mg/m² temozolamide. He subsequently underwent 5 cycles of temozolamide to 200 mg/m².

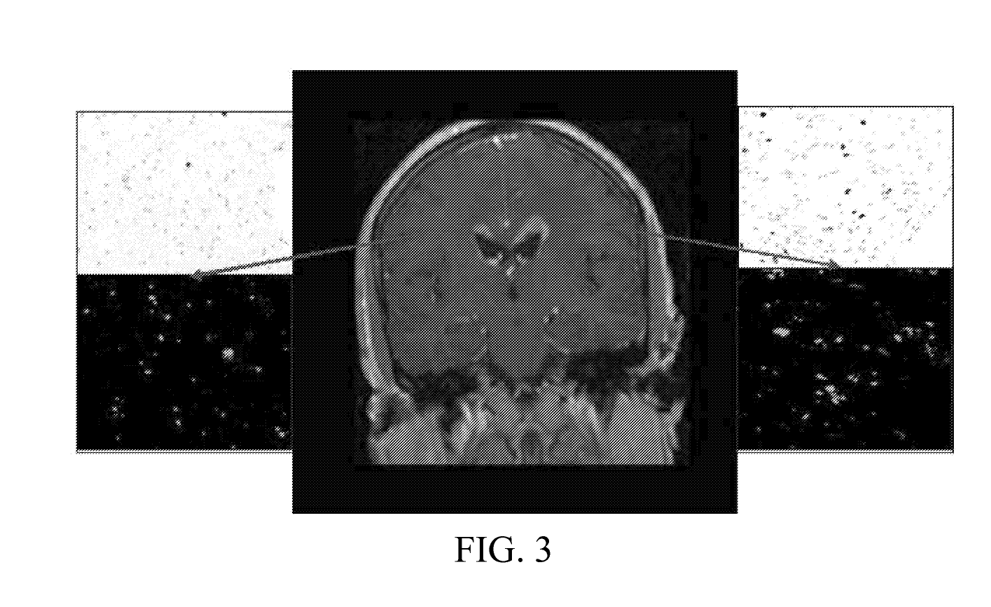

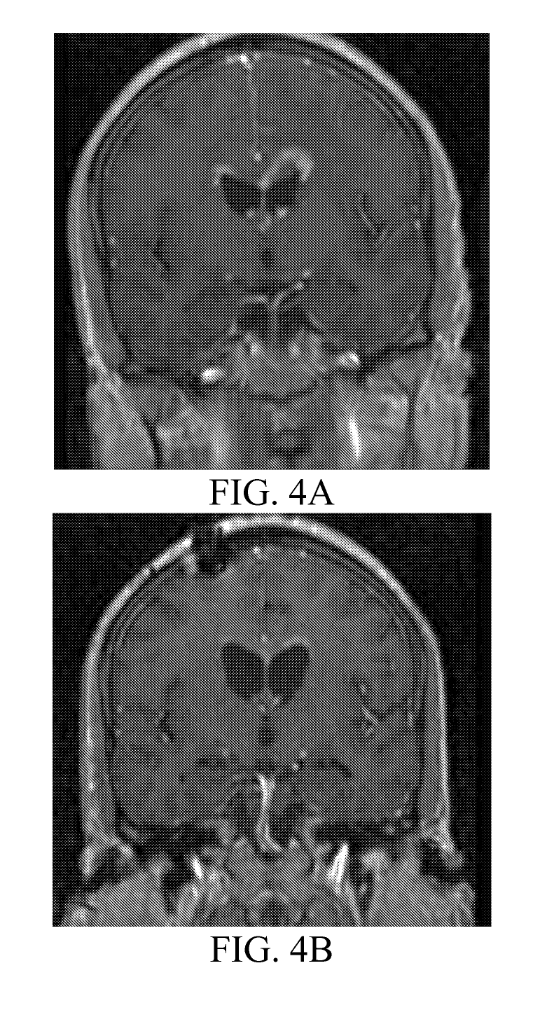

Twenty-four weeks later, MR imaging showed local tumor recurrence in the right frontal lobe with extension into the corpus callosum to the contralateral side (FIGS. 3, and 4A). The patient's KPS was 90 and underwent biopsy of the bilateral periventricular lesions, and placement of Ommaya reservoir. Pathology results demonstrated GBM in the left-sided periventricular area, GFAP+ with a Ki-67 labeling index of 10% (FIG. 3), and hypercellularity of the right frontal periventricular area, GFAP+ with a Ki-67 labeling index of approximately 2% (FIG. 3). Both biopsy specimens demonstrated intense nestin immunoreactivity (FIG. 3).

### Liposomal Encapsulated Ara-C (DepoCyt®) Treatment

The patient underwent both induction and consolidation phases of IT DepoCyt® (cycles 1-6) and is currently undergoing cycle 7 (11). Treatment related toxicity has been limited to severe headaches beginning 6-7 days after finishing the 5 day course of dexamethasone during each cycle. This has been treated using a combination of intermittent Naproxyn 500 mg BID as needed, and/or 5 day methylprednisolone tapering doses. No other treatment-related complications have occurred. MRI obtained after the induction phase demonstrates diminished periventricular enhancement bilaterally (FIG. 4A, B).

### Example 2

### DepoCyt® Therapy Protocol

### Induction Phase

Patients undergo an induction phase of intrathecal (IT) DepoCyt®, using 50 mg every two weeks (+/−3 days) for one month (Cycles 1, 2). DepoCyt® is steriley administered over 5 minutes into the Ommaya reservoir via 25 gauge butterfly needle. Prior to injection the intracranial pressure (ICP) is checked and 7 cc's of CSF are slowly removed. Following administration of DepoCyt®, the Ommaya is flushed with 2 of the 7 mls of CSF initially removed. All patients receive dexamethasone 4 mg three times a day on days 1-5 of each cycle.

Evaluation of the therapeutic response is assessed clinically by a physical exam at each visit prior to further IT DepoCyt® treatment, and systemic toxicity utilizing hematological and blood chemistry analysis is again assessed prior to all DepoCyt® doses. DepoCyt® is withheld if there is evidence of systemic toxicity and not restarted until return to baseline values.

Clinical failure prompts immediate MRI evaluation, and further IT DepoCyt® is withheld until this assessment is made.

The intracranial pressure (ICP) is recorded prior to DepoCyt® injection via accessing the Ommaya reservoir with a butterfly needle attached to a manometer with the patient supine. Elevated ICP (>20 cm H2O) is documented and an MRI is obtained to rule out hydrocephalus, and/or tumor recurrence.

Brain MRI is obtained after the induction phase. Patients enter the consolidation phase (below) if there is evidence of stable disease clinically and radiographically.

In addition to the history and physical examination with comprehensive neurological examination and hematological and blood chemistry analysis prior to each DepoCyt® injection, the patient undergoes a battery of tests including: neuropsychiatry examination, assessment of activities of daily living (ADLs), and quality of life measures prior to Cycle 2.

If a scheduled study dose is missed, the dose is given as soon as appropriate. Following this dose, the patient resumes the protocol schedule.

### Consolidation Phase

Patients with stable disease (clinically and radiographically), not exhibiting systemic toxicity, undergo a three month consolidation phase of IT DepoCyt®, using 50 mg every two weeks (+/−3 days) for one month (Cycles 3, 4), followed by 50 mg IT DepoCyt® every four weeks (+/−3 days) for two months (Cycles 5, 6). All patients also receive dexamethasone 4 mg TID on days 1-5 of each cycle.

Evaluation of the therapeutic response is assessed clinically by physical exam at each visit prior to further IT DepoCyt® treatment, and systemic toxicity utilizing hematological and blood chemistry analysis is again assessed prior to all DepoCyt® doses. DepoCyt® is withheld if there is evidence of systemic toxicity and not restarted until return to baseline values.

Clinical failure prompts immediate MRI evaluation, and further IT DepoCyt® is withheld until this assessment is made. The ICP is recorded prior to DepoCyt® injection. Elevated ICP (>20 cm H2O) is documented and an MRI is obtained to rule out hydrocephalus, and/or tumor recurrence.

Brain MRI is obtained after the Cycles 4 and 6 of the consolidation phase. Patients enter the maintenance phase (below) if there is evidence of stable disease clinically and radiographically.

In addition to the history and physical examination with comprehensive neurological examination and hematological and blood chemistry analysis prior to each DepoCyt® injection, the patient undergoes a battery of tests including: neuropsychiatry examination, assessment of activities of daily living (ADLs), and quality of life measures prior to Cycles 4 and 6. If a scheduled study dose is missed, the dose is given as soon as appropriate. Following this dose, the patient resumes the protocol schedule.

### Maintenance Phase

Patients without progression or toxicity undergo maintenance therapy using 50 mg IT DepoCyt® every four weeks (+/−3 days) for a maximum of 8 months (cycles 7-14) or until recurrence or toxicity ensues. The first maintenance is given 4 weeks after the last consolidation dose. All patients receive 4 mg dexamethasone three times a day on days 1-5 of each cycle.

Evaluation of the therapeutic response is assessed clinically by physical exam at each visit prior to further IT DepoCyt® treatment, and systemic toxicity utilizing hematological and blood chemistry analysis is again assessed prior to all DepoCyt® doses. DepoCyt® is withheld if there is evidence of systemic toxicity and not restarted until return to baseline values. The treatment continues without interruption as long as there is no tumor recurrence or progression and toxicity is acceptable.

The ICP is recorded prior to DepoCyt® injection. Elevated ICP (>20 cm H2O) is documented and an MRI is obtained to rule out hydrocephalus, and/or tumor recurrence. Brain MRI is obtained after the Cycles 9, 12, and 14 of the maintenance phase. After 52 weeks of therapy, patients continue maintenance therapy after that time at the discretion of their treating physicians.

In addition to the history and physical examination with comprehensive neurological examination and hematological and blood chemistry analysis prior to each DepoCyt® injection, the patient undergoes a battery of tests including: neuropsychiatry examination, assessment of activities of daily living (ADLs), and quality of life measures prior to Cycles 9, 12, and 14. If a scheduled study dose is missed, the dose is given as soon as appropriate. Following this dose, the patient resumes the protocol schedule.

Dose limiting toxicities (DLT) are graded according to The NCI Common Terminology Criteria for Adverse Events v3.0 (CTCAE) (Appendix II) where a severity grading scale is provided for each adverse event (AE) term. If multiple toxicities are encountered, the presence of DLT is based on the most severe toxicity experienced. DLT will be defined as any of the following events occurring during treatment with IT DepoCyt®: any grade 3 or 4 thrombocytopenia, anemia and/or neutropenia, or any non-hematologic grade 3 or 4 toxicity.

Throughout this application, various publications are referenced. The disclosures of these publications in their entireties are hereby incorporated by reference into this application in order to more fully describe the state of the art to which this invention pertains.

### REFERENCES

1. F. G. Davis, S. Freels, J. Grutsch, S. Barlas, S. Brem, J Neurosurg 88, 1 (January, 1998).
2. D. F. Deen et al., J Neurooncol 16, 243 (June, 1993).
3. P. Kleihues, L. H. Sobin, Cancer 88, 2887 (Jun. 15, 2000).
4. K. E. Wallner, J. H. Galicich, G. Krol, E. Arbit, M. G. Malkin, Int J Radiat Oncol Biol Phys 16, 1405 (June, 1989).
5. F. G. Barker, 2nd et al., Neurosurgery 42, 709 (April, 1998).
6. B. Young et al., J Neurosurg 55, 917. (December, 1981).
7. F. Paulsen et al., J Cancer Res Clin Oncol 125, 411 (July, 1999).
8. H. Brem et al., J Neurooncol 26, 111 (November, 1995).
9. H. Brem et al., Lancet 345, 1008 (Apr. 22, 1995).
10. A. K. Choucair et al., J Neurosurg 65, 654 (November, 1986).
11. M. J. Glantz et al., Clin Cancer Res 5, 3394 (November, 1999).
12. R. Galli et al., Cancer Res 64, 7011 (Oct. 1, 2004).
13. K. A. Jaeckle et al., J Neurooncol 57, 231 (May, 2002).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1. A method for the treatment of a subject with glioma, comprising: administering to the subject an effective amount of cytarabine and a diuretic.

2. The method of claim 1, wherein the cytarabine is liposomal cytarabine (DepoCyt®).

3. The method of claim 2, wherein the glioma is Glioblastoma Multiforme, is recurrent Glioblastoma Multiforme, astrocytoma grade I, astrocytoma grade II, astrocytoma grade III, astrocytoma grade IV, ependymoma, oligodendroglioma, brainstem glioma, and mixed glioma.

4-5. (canceled)

6. The method of claim 2, wherein the effective amount is administered during one or more treatment sessions.

7. The method of claim 6, wherein the sum dosage of DepoCyt® administered over all treatment sessions is at least about 20 milligrams.

8. The method of claim 6, wherein the sum dosage administered over all treatment sessions is at least about 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, or 750 milligrams or greater.

9. The method of claim 6, wherein the sum dosage of DepoCyt® administered over all treatment sessions is greater than about 450 milligrams.

10. The method of claim 6, wherein the effective amount of DepoCyt® administered to the subject at each treatment session is at least about 20 milligrams.

11. The method of claim 10, wherein the effective amount of DepoCyt® administered to the subject at each treatment session is at least about 30, 40, or 50 milligrams or greater.

12. The method of claim 6, wherein the effective amount of DepoCyt® is administered in a solution having a concentration of about 20 milligrams DepoCyt® or less in each milliliter of solution.

13. The method of claim 6, wherein the effective amount of DepoCyt® is administered in a solution having a concentration of about 10 milligrams of DepoCyt® or less in each milliliter of solution.

14. The method of claim 6, wherein the treatment sessions are completed in at least about one month.

15. The method of claim 14, wherein the treatment sessions are completed in at least about 2, 3, 4, 5, 6, 7, 8, 9, 10, 11, 12, 13, 14, or 15 months or longer.

16. The method of claim 6, wherein the treatment sessions comprise administering at least about 20 milligrams of DepoCyt® to the subject at each of a first and second treatment sessions.

17. The method of claim 16, wherein the first and second treatment sessions are temporally separated by about two weeks.

18. The method of claim 16, further comprising administering at least about 20 milligrams of DepoCyt® to the subject at each of a third, fourth, fifth and sixth treatment session.

19. The method of claim 18, wherein each of the third, fourth, fifth and sixth treatment sessions are temporally separated by at least about two weeks.

20. The method of claim 19, wherein each of the fifth and sixth treatment sessions are temporally separated by about four weeks.

21. The method of claim 18, further comprising administering at least about 20 milligrams of DepoCyt® to the subject at a seventh, eighth, ninth, tenth, eleventh, twelfth, thirteenth, and fourteenth or more treatment session.

22. The method of claim 21, wherein each of the seventh, eighth, ninth, tenth, eleventh, twelfth, thirteenth, and fourteenth or more treatment sessions are temporally separated by about four weeks.

23. The method of claim 2, wherein the diuretic is acetazolamide (Diamox), furosemide.

24. (canceled)

25. The method of claim 2, wherein the diuretic is administered orally, intravenously, intrathecally, intraventricularly, wherein the diuretic is administered preceding or following DepoCyt® administration.

26-29. (canceled)

30. The method of claim 2, wherein the DepoCyt® is administered intrathecally, by an intrathecal catheter attached to a subcutaneous port, by spinal puncture needle, or by temporary catheter placement, intraventricularly, using an Ommaya reservoir, ventricular shunt, or ventricular shunt reservoir, wherein the DepoCyt® is injected directly into the tumor or surrounding parenchyma using an implanted reservoir catheter system placed into the tumor or resected tumor bed is directly infused into the tumor, tumor bed, or surrounding brain parenchyma using a catheter attached to an implanted or external pump, wherein the DepoCyt® is infused by convection enhanced delivery.

31-36. (canceled)

37. The method of claim 2, wherein greater than 9 sessions of about 50 milligrams of DepoCyt® are administered to the subject.

38. The method of claim 37, wherein the administration of each session is separated by about two to four weeks.

39. The method of claim 2, wherein an amount of spinal fluid is removed prior to administration of DepoCyt®.

40. A method for the treatment of a subject with recurrent Glioblastoma Multiforme, comprising administering to the subject an effective amount of cytarabine.

41-69. (canceled)

70. A method for the treatment of a subject with glioma, comprising administering an effective amount of cytarabine and one or more other anti-cancer drugs or anti-cancer therapies.

71-104. (canceled)

105. A method for the treatment of a subject with glioma, comprising administering an effective amount of cytarabine in a predetermined fluid volume into one or more ventricles of the subject's brain and allowing the effective amount of cytarabine to remain within the subject to effect treatment of the glioma.

106-114. (canceled)

115. A method for the treatment of a subject with glioma, comprising intrathecally administering an effective amount of cytarabine and allowing the effective amount of cytarabine to remain within the subject to treat the glioma.

116-123. (canceled)

124. A composition comprising a mixture of DepoCyt® and a diuretic.

125-129. (canceled)
